(19) United States
(12) Patent Application Publication
(10) Pub. No.: US 2002/0001856 A1
(43) Pub. Date: Jan. 3, 2002

(54) Methods and devices for achieving long incubation times in high-throughput systems

(76) Inventors: Andrea W. Chow, Los Altos, CA (US); Anne R. Kopf-Sill, Portola Valley, CA (US)

Correspondence Address: LAW OFFICES OF JONATHAN ALAN QUINE, P O BOX 458, ALAMEDA, CA, 94501

(21) Appl. No.: 09/825,090

(22) Filed: Apr. 2, 2001

Related U.S. Application Data
(60) Provisional application No. 60/195,591, filed on Apr. 6, 2000.

(51) Int. Cl. G01N 33/536; C12M 1/34; G06F 19/00; G01N 33/48; G01N 33/50

(52) U.S. Cl. 436/536; 435/287.2; 702/19

(57) ABSTRACT

Methods of performing high throughput screening, e.g., for assays with long incubation times and/or reaction times, in a microfluidic device are provided. The methods combine the use of multiple channels with serially loading to provide high throughput and long incubations. Also included are microfluidic devices and integrated systems for performing high throughput long incubation assays as well as methods for designing devices for use in such assays.

### CROSS-REFERENCES TO RELATED APPLICATIONS

Pursuant to 35 U.S.C. §§ 119 and/or 120, and any other applicable statute or rule, this application claims the benefit of and priority to U.S. Ser. No. 60/195,591, filed on Apr. 6, 2000, the disclosure of which is incorporated by reference.

### COPYRIGHT NOTIFICATION

Pursuant to 37 C.F.R. § 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

When carrying out chemical or biochemical analyses, assays, syntheses, or preparations one performs a large number of separate manipulations on the material or component to be assayed, including measuring, aliquotting, transferring, diluting, mixing, separating, detecting, incubating, etc. Microfluidic technology miniaturizes these manipulations and integrates them so that they can be performed within one or a few microfluidic devices.

For example, new and faster microfluidic methods of performing biological assays in microfluidic systems have been developed, such as those described by the pioneering application of Parce et al., “High Throughput Screening Assay Systems in Microscale Fluidic Devices” U.S. Pat. No. 5,942,443 and in Knapp et al., “Closed Loop Biochemical Analyzers” (WO 98/45481; PCT/US98/06723). For example, high throughput methods for analyzing biological reagents, including proteins, are described in these applications.

For some bioassays, a constant flow of material is useful to maintain a fixed assay reaction time. Therefore, the ability to modulate a flow rate and obtain constant incubation and reaction times in a microfluidic system when performing dilutions is useful to the integration of fluidic sample and reagent manipulations in a microfluidic assay format. For example, Kopf-Sill et al. describe methods of providing constant flow rates in “Dilutions in High Throughput Systems with a Single Vacuum Source,” U.S. Ser. No. 60/150,670. Therefore, methods exist to provide constant incubation times.

High throughput methods and microfluidic systems with long incubation times would be a useful addition to the art. Various assays use long incubation times and/or reaction times, such as kinase reactions, fluorogenic enzyme assays, and the like.

Improved methods and microfluidic systems for providing long incubation times are, accordingly, desirable, particularly those which take advantage of high-throughput, low cost microfluidic systems. The present invention provides these and other features by providing high throughput microscale systems for providing high throughput assays for systems with long incubation and/or reaction times and many other features that will be apparent upon complete review of the following disclosure.

### SUMMARY OF THE INVENTION

The present invention provides methods and devices for high-throughput assays with long incubation times. Long incubation times are achieved in a high-throughput manner using channel configurations that allow loading and incubation of samples concurrent with detection and off-loading of other samples. For example, while one sample or set of samples is detected, another sample or set of samples is loaded and/or incubated or reacted. The channel configurations typically comprise parallel assay or incubation channels that are used to park samples for extended periods of time while a reaction proceeds. Alternatively, the samples may be flowed slowly through the channel instead of parked, i.e., in a stationary manner, in the channels. Serial injections and fluid flow are switched between parallel channels to detect samples that have completed reaction while allowing other samples to continue to undergo reaction in a parallel channel.

In one aspect, methods of designing microfluidic devices are provided. The methods comprise selecting a desired number of samples for screening, selecting an incubation or reaction time for the desired number of samples, and selecting a sample plug length. The sample plug length is typically at least as long as the incubation time multiplied by the thermal diffusivity and Taylor dispersion of the samples. Therefore, the method also optionally includes calculating the thermal diffusivity and/or dispersion of the sample.

A plurality of interconnected channels is provided to accommodate the desired number of samples for the desired incubation or reaction time. The interconnected channels typically have a combined length substantially equal to the desired number of samples multiplied by the sample plug length. If buffers are included in the screening, the combined length is substantially equal to the desired number of samples multiplied by the sample plug length plus the buffer plug length.

The desired number of samples selected is typically between about 10 samples and about 1000 samples or more, preferably between about 20 samples and about 500 samples. Typically the desired number of samples is between about 40 samples and about 250 samples or between about 80 samples and about 150 samples. Incubation times are typically between about 0.5 minutes and about 1 hour or more, more typically between about 5 minutes and about 30 minutes. Sample plug lengths optionally range from about 50 μm to about 5 mm. The sample plug lengths optionally vary depending on the channel length, incubation time and diffusion and/or dispersion such that lengths outside these ranges are optionally used. Typical ranges are from about 100 μm to about 5 mm, more typically about 500 μm to about 3 mm or about 850 μm to about 1 mm.

Typically, devices are designed to comprise about 2 to about 50 analysis or incubation channels, more typically from about 2 to about 30 channels. Preferably the number of channels ranges from about 4 to about 20 channels or about 6 to about 10 channels. The channels provided typically range in length from about 20 mm to about 2000 mm, preferably from 40 mm to about 200 mm.

Devices designed according to the guidelines above typically provide sampling rates between about 60 seconds per sample and about 6 seconds per sample, providing high throughput screening for assays involving long incubation or reaction times.

Devices of the invention typically comprise at least one sample source, e.g., comprising the desired number of samples and or buffers, a plurality of channels, e.g., incubation or analysis channels, fluidly coupled to the at least one sample source; and one or more detection channel regions, fluidly coupled to the channels. The channels are typically structurally configured or designed, as described above, to provide an incubation time of at least about 10 minutes with a throughput of about 1 sample about every 60 seconds or less. For example, a typical device comprises four channels, which four channels each comprise a length of about 20 mm to about 100 mm, preferably about 50 mm to about 100 mm. Alternative devices comprise six channels of about 20 mm to about 100 mm, typically about 50 mm to about 80 mm. In other embodiments, the devices comprise 2 channels of about 50 mm to about 200 mm, typically about 100 mm to about 160 mm. Channel ranges also vary quite a bit and are chosen to optimize channel parameters for the desired incubation time. Thus, other channel lengths not listed here are possible.

The devices also optionally comprise fluid control elements and detectors as described above. For example a fluid direction system in a microfluidic system comprises fluid control elements. During operation of the system, the fluid control elements direct movement of the samples, e.g., into the channels and/or detection channel regions. Various fluid control elements are optionally fluidly coupled to the plurality of channels for loading and unloading the samples from the devices of the present invention and for directing the movement of the samples through the channels. For example each channel is typically coupled to at least one fluid control element. The fluid control elements optionally comprise pressure sources, vacuum sources, electrokinetic controllers, and the like. In some embodiments a single fluid control element, e.g., a vacuum, is coupled to all of the channels. For example a single control element is optionally coupled to a valve manifold comprising one or more electronically controlled valves, e.g., solenoid valves.

For example, during operation of the system, the fluid direction system directs the movement of at least a first sample into the channels, e.g., incubation channels, concurrent with directing at least a second sample into a detection channel region. In addition, the fluid direction system directs the movement of at least a first sample into the channels concurrent with incubating at least a second sample and directs the movement of at least one sample into a detection channel region concurrent with incubating one or more additional samples. Therefore, the loading, incubating and detecting are carried out concurrently, allowing high sample throughput while maintaining long incubation times for each sample. One sample is loaded and then incubates while a second sample, a third sample and so forth are loaded. Once all samples are loaded, detection begins on the first sample loaded, which first sample has been incubating throughout the process. Once detected, the samples are optionally off-loaded or transported to a waste well, providing space in the channels to load additional samples as each completed sample is off-loaded to a waste well.

During operation of the system, the fluid direction system directs movement of at least one member of the plurality of samples into the plurality of channels and at least one member of the plurality of samples into a detection region about every 60 seconds or less, about every 40 seconds or less, about every 30 seconds or less, about every 10 seconds or less, or about every 6 seconds or less. Between the time a sample is loaded and the time the sample is detected, the sample remains in the channel, e.g., incubating or reacting, about 5 minutes to about 50 minutes. More typically, the samples remain in the channels from about 10 minutes to about 30 minutes. Buffers are optionally transported through the system in addition to the samples. For example, the fluid direction system optionally directs movement of a buffer into the plurality of channels after movement of each member of the plurality of samples into the plurality of channels.

The detection system typically comprises one or more detectors, e.g., fluorescent detectors, positioned proximal to at least one of the one or more detection channel regions. In one embodiment, a single detector is positioned proximal to substantially all of the detection channel regions.

In another aspect, methods of incubating a plurality of samples in a high-throughput microfluidic system are provided. The methods comprise loading a plurality of samples into a plurality of channels, e.g., in a device described above. Each member of the plurality of samples is incubated or reacted in the plurality of channels, e.g., parallel incubation channels, for at least about 5 minutes. After incubation or reaction, the samples are flowed through one or more detection region. At least one member of the plurality of samples is loaded into at least one of the plurality of channels about every 60 seconds or less, thereby achieving a long incubation time for substantially all of the samples in the plurality of samples, while concomitantly processing the plurality of samples in a high-throughput format. The methods are typically carried out in a device designed as described above.

In one embodiment, loading the samples into the device comprises dividing the plurality of samples into a plurality of portions and loading each of the portions into a different member of the plurality of channels. For example, the plurality of samples is optionally divided into four portions or groups, each of which is loaded into a different channel. For example, in a device comprising about 4 channels and about 40 samples, about 10 samples are typically flowed into each of the four channels. Typically each channel is loaded in about 100 to about 500 seconds, more typically in about 200 seconds to about 300 seconds. The movement of the samples is directed by fluid control elements as described above.

The samples are typically detected in a detection region or channel before being unloaded from the device. Detection typically comprises fluorescently detecting the plurality of samples, e.g., detecting at least one sample about every 60 seconds or less, about every 40 seconds or less, about every 20 seconds or less, about every 10 seconds or less, or about every 6 seconds or less.

Therefore as one sample is loaded another is detected, and all the samples loaded in between remain in the channels to incubate. Loading and detecting are optionally performed continuously or are iteratively repeated, thus providing high-throughput screens for assays involving long-incubation times and processing, e.g., thousands of compounds or reactions, e.g., in a day.

### BRIEF DESCRIPTION OF THE FIGURES

FIG. 1: Panels A, B, and C are schematic drawings of an integrated system of the invention, including a body structure, microfabricated elements, and a pipettor channel.

FIG. 2: Schematic drawing of an integrated system of the invention further depicting incorporation of a microwell plate, a computer, detector and a fluid direction system. The integrated system is optionally used with either the device or body structure of FIG. 3, 4, or any other suitable microfluidic device.

FIG. 3: Microfluidic channel configuration comprising four parallel incubation or reaction channels.

FIG. 4: Multiplexed set of channels in which multiple sippers are connected to the same pressure/vacuum controller. The fluid control element comprises four discreet fluid control elements, each of which is fluidly coupled to at least one channel from each sipper.

### DETAILED DISCUSSION OF THE INVENTION

The present invention provides methods and devices used to perform high throughput screening on systems with long incubation and/or reaction times. For example, fluorogenic enzyme assays, binding assays, non-fluorogenic enzyme assays, kinase assays, cell based assays, in-line PCR reactions, and the like all use long incubation or reaction times. The present invention provides devices for performing these assays, methods of designing such devices, and methods for performing the assays.

The devices used to provide long incubation times in high throughput format typically comprise a plurality of analysis or incubation channels, e.g., parallel incubation channels. Samples are serially loaded into the channels and parked or incubated therein. Parking optionally comprises leaving the samples in the same location in the channels, e.g., the incubation channels, or moving the samples through the channels at a slow flow rate, so that they remain in the channels for a long incubation time. The samples are then serially unloaded, e.g., to a detection region or waste well. Each sample incubates while others are loaded and/or unloaded or detected, thereby providing each sample with a long incubation while maintaining a throughput of samples of about 1 sample about every 60 seconds or less. The throughput is maintained by continuously loading, incubating and unloading samples into and from the incubation channels of the device.

For example, the microfluidic devices presented typically comprise from about 2 to about 50 analysis or incubation channels. A plurality of samples is divided into portions and one portion of samples is loaded into each channel. The first sample loaded into a channel begins to incubate and continues to incubate and/or react until all samples are loaded into the channels. By the time loading is completed, the first sample has typically been incubating, e.g., for about 5 minutes to about 30 minutes. The amount of time the samples incubate depends on the load time, the number of channels loaded, and the number of samples loaded per channel. For example when 40 samples are loaded into four channels with 24 seconds used to load each sample, the first sample incubates for 16 minutes, i.e., the time it remains in the channel while all the other samples are loaded. The samples are then serially unloaded, e.g., into a detection region and detected, at the same rate, thus achieving the same incubation time for each sample. The following samples achieve the same incubation time, e.g., by remaining parked in an incubation channel or moving slowly through the channel as the other samples are loaded, unloaded, and/or detected.

I. Microfluidic Devices of the Invention—Generally

Microfluidic devices generally comprise a body structure with microscale channels disposed therein. For example, the present system typically comprises two or more channels, e.g., parallel channels. The channels are fluidly coupled to each other and to various reservoirs or other sources of fluidic materials, e.g, sample sources, substrate sources, enzyme sources, waste wells, and the like. Materials used in the present invention include, but are not limited to, buffers, diluents, substrate solutions, enzyme solutions, and sample solutions. In addition, the channels optionally comprise detection regions.

For example, various channels and channel regions are disposed throughout the microfluidic device. The devices optionally include a main channel region into which a sample is introduced. For example, a sample containing a potential modulator or activator of an enzyme of interest is introduced into a main channel, e.g., through a sipper capillary. An assay to determine the effect of the modulator, e.g., an activator or an inhibitor, on the enzyme's reaction rate is then optionally performed by allowing the enzyme to react with a substrate in the presence of the modulator. For example, enzyme and substrate materials are optionally flowed into a main channel to contact a modulator and react. These materials are optionally flowed into the main channel from side channels, e.g., side channels coupled to reservoirs. Many reactions or assays of interest involve long incubation times, e.g., to allow time for the enzyme and substrate to react, e.g., in the presence of an inhibitor. A “long incubation time” in the present invention refers to an incubation time of about 0.5 minutes to about 1 hour. Typically, “long” incubation times are about 5 minutes to about 50 minutes, more typically about 10 minutes to about 30 minutes. Incubation time or reaction time refers to the time in which two or more reactants are allowed to mix and/or react with each other, e.g., an enzyme and substrate are allowed to mix and react to form a product. These long incubations, e.g., of enzyme and substrate, are achieved in the present invention by parking or positioning sample plugs in incubation or assay channels. For example, after mixing with inhibitor in the main channel, enzyme and substrate are flowed into an incubation channel, e.g., one of a set of parallel incubation or assay channels. In the incubation channel, the sample is optionally parked in a stationary position or flowed slowly through the channel such that a long incubation is obtained before the sample is detected. For example, while one sample or a series of samples are parked in one of a set of parallel incubation channels, other samples are loaded, e.g., into a second parallel incubation channel. While some samples are loaded or unloaded, others remain parked in the incubation channels, i.e., reacting or incubating for the desired time.

The channels of the present invention are structurally configured to allow long incubation times in a high throughput format. A “structurally configured” channel is one that is configured to provide a desired result. Typically, the channels in the present invention are configured by adjusting the length, width, fluidic resistance, number, or distribution of channels in the device. For example the number of channels ranges from about 2 channels to about 16 channels. The channels are typically distributed in a parallel fashion in which each channel is fluidly coupled to a waste well and a source of one or more reagents. In addition, each channel is typically fluidly coupled to a sample capillary for introducing samples into the device and one or more detection region for detecting the samples. The arrangement and length of the channels in the present invention is configured to allow a desired number of samples to be incubated within the device at one time to provide long incubation times for multiple samples. At the same time, the channels are loaded, e.g., from a microwell plate, and unloaded, e.g., after detection, at a rate of about 1 sample about every 60 seconds or less, about every 40 seconds or less, about every 20 seconds or less, about every 20 seconds or less, or about every 6 seconds or less. Alternative methods for configuring the channels include, but are not limited to, varying the channel length or cross section and/or adding a flow-retarding matrix. These alternative changes typically affect flow rate. For more detail on structurally configuring channels for desired flow rates using channel length and dimensions, see, e.g., U.S. Ser. No. 09/238,467, “Devices, Systems and Methods for Time Domain Multiplexing of Reagents,” filed Jan. 28, 1999 by Chow et al.

The reservoirs or wells of the present invention are locations at which samples, components, reagents and the like are added into the device for assays to take place. Introduction of these elements into the system is carried out as described below. The reservoirs are typically placed so that the sample or reagent is added into the system upstream from the location at which it is used. For example, a dilution buffer is added upstream from the source of a regent if the sample is to be diluted before reaction with the reagent. Alternatively, waste wells or reservoirs are used to store samples after a reaction or assay has been completed. In the present invention, samples for which the screening or assay has been completed are optionally off-loaded, e.g., into a waste well. The removal of the completed samples provides space in the channels to load and incubate other samples. In this fashion, the devices of the invention are optionally used in a high throughput manner.

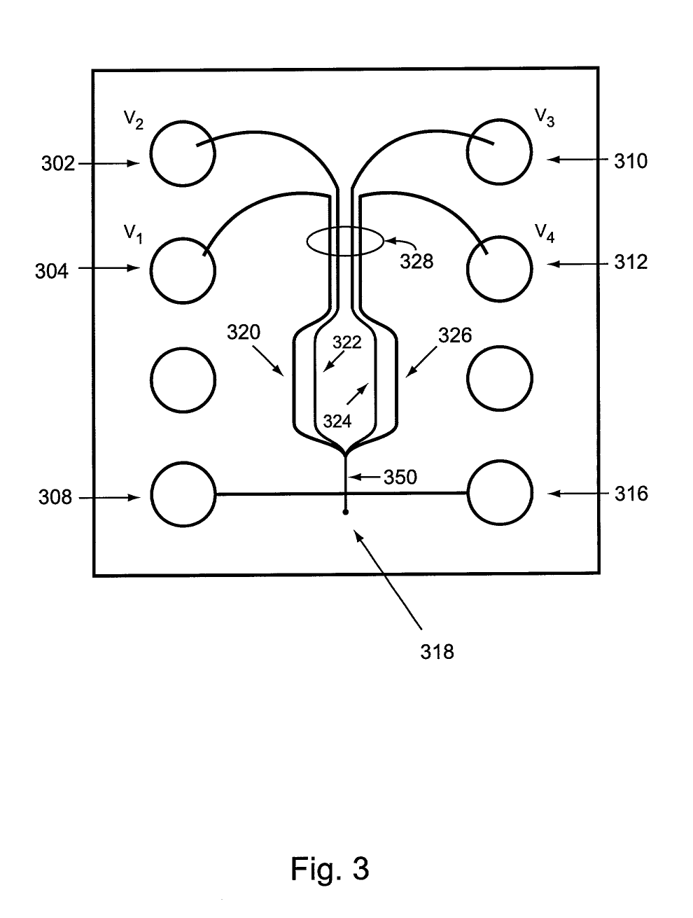

Detection regions are also included in the present devices. The detection region is optionally a subunit of a channel or of multiple channels that are close in space, or it optionally comprises a distinct channel that is fluidly coupled to the plurality of channels in the microfluidic device. In the present invention one detection region is typically located at a position that is proximal to each of the channels, e.g., incubation channels. For example, in FIG. 3, since the channels are configured to converge in one area, detection region 328 is positioned proximal to channels 320, 322, 324, and 326. Alternatively, multiple detection regions are optionally located proximal to each of the waste wells in FIG. 3, such as wells 302, 304, 310, 312, and the like.

The detection window or region at which a signal is monitored typically includes a transparent cover allowing visual or optical observation and detection of the assay results, e.g., observation of a colorimetric or fluorescent signal or label. Such regions optionally include one or more detectors. Examples of suitable detectors for use in the detection regions are well known to those of skill in the art and are discussed in more detail below.

The elements described above, including but not limited to, incubation channels, reaction channels, detection regions, and reservoirs are optionally combined into microfluidic devices that are useful in performing high-throughput screening, e.g, fluorogenic enzyme inhibition assays in which long incubation times are used. Specific examples of channel configurations and how to design them are provided below and in the figures. Other possible configurations using substantially the same elements will be apparent upon review of the entire disclosure.

A variety of microscale devices are optionally adapted for use in the present invention, e.g., by designing and configuring the channels as discussed below. These devices are described in various PCT applications and issued U.S. patents by the inventors and their coworkers, including U.S. Pat. No. 5,699,157 (J. Wallace Parce) issued Dec. 16, 1997, U.S. Pat. No. 5,779,868 (J. Wallace Parce et al.) issued Jul. 14, 1998, U.S. Pat. No. 5,800,690 (Calvin Y. H. Chow et al.) issued Sep. 1, 1998, U.S. Pat. No. 5,842,787 (Anne R. Kopf-Sill et al.) issued Dec. 1, 1998, U.S. Pat. No. 5,852,495 (J. Wallace Parce) issued Dec. 22, 1998, U.S. Pat. No. 5,869,004 (J. Wallace Parce et al.) issued Feb. 9, 1999, U.S. Pat. No. 5,876,675 (Colin B. Kennedy) issued Mar. 2, 1999, U.S. Pat. No. 5,880,071 (J. Wallace Parce et al.) issued Mar. 9, 1999, U.S. Pat. No. 5,882,465 (Richard J. McReynolds) issued Mar. 16, 1999, U.S. Pat. No. 5,885,470 (J. Wallace Parce et al.) issued Mar. 23, 1999, U.S. Pat. No. 5,942,443 (J. Wallace Parce et al.) issued Aug. 24, 1999, U.S. Pat. No. 5,948,227 (Robert S. Dubrow) issued Sep. 7, 1999, U.S. Pat. No. 5,955,028 (Calvin Y. H. Chow) issued Sep. 21, 1999, U.S. Pat. No. 5,957,579 (Anne R. Kopf-Sill et al.) issued Sep. 28, 1999, U.S. Pat. No. 5,958,203 (J. Wallace Parce et al.) issued Sep. 28, 1999, U.S. Pat. No. 5,958,694 (Theo T. Nikiforov) issued Sep. 28, 1999, U.S. Pat. No. 5,959,291 (Morten J. Jensen) issued Sep. 28, 1999, U.S. Pat. No. 5,964,995 (Theo T. Nikiforov et al.) issued Oct. 12, 1999, U.S. Pat. No. 5,965,001 (Calvin Y. H. Chow et al.) issued Oct. 12, 1999, U.S. Pat. No. 5,965,410 (Calvin Y. H. Chow et al.) issued Oct. 12, 1999, U.S. Pat. No. 5,972,187 (J. Wallace Parce et al.) issued Oct. 26, 1999, U.S. Pat. No. 5,976,336 (Robert S. Dubrow et al.) issued Nov. 2, 1999, U.S. Pat. No. 5,989,402 (Calvin Y. H. Chow et al.) issued Nov. 23, 1999, U.S. Pat. No. 6,001,231 (Anne R. Kopf-Sill) issued Dec. 14, 1999, U.S. Pat. No. 6,011,252 (Morten J. Jensen) issued Jan. 4, 2000, U.S. Pat. No. 6,012,902 (J. Wallace Parce) issued Jan. 11, 2000, U.S. Pat. No. 6,042,709 (J. Wallace Parce et al.) issued Mar. 28, 2000, U.S. Pat. No. 6,042,710 (Robert S. Dubrow) issued Mar. 28, 2000, U.S. Pat. No. 6,046,056 (J. Wallace Parce et al.) issued Apr. 4, 2000, U.S. Pat. No. 6,048,498 (Colin B. Kennedy) issued Apr. 11, 2000, U.S. Pat. No. 6,068,752 (Robert S. Dubrow et al.) issued May 30, 2000, U.S. Pat. No. 6,071,478 (Calvin Y. H. Chow) issued Jun. 6, 2000, U.S. Pat. No. 6,074,725 (Colin B. Kennedy) issued Jun. 13, 2000, U.S. Pat. No. 6,080,295 (J. Wallace Parce et al.) issued Jun. 27, 2000, U.S. Pat. No. 6,086,740 (Colin B. Kennedy) issued Jul. 11, 2000, U.S. Pat. No. 6,086,825 (Steven A. Sundberg et al.) issued Jul. 11, 2000, U.S. Pat. No. 6,090,251 (Steven A. Sundberg et al.) issued Jul. 18, 2000, U.S. Pat. No. 6,100,541 (Robert Nagle et al.) issued Au. 8, 2000, U.S. Pat. No. 6,107,044 (Theo T. Nikiforov) issued Aug. 22, 2000, U.S. Pat. No. 6,123,798 (Khushroo Gandhi et al.) issued Sep. 26, 2000, U.S. Pat. No. 6,129,826 (Theo T. Nikiforov et al.) issued Oct. 10, 2000, U.S. Pat. No. 6,132,685 (Joseph E. Kersco et al.) issued Oct. 17, 2000, U.S. Pat. No. 6,148,508 (Jeffrey A. Wolk) issued Nov. 21, 2000, U.S. Pat. No. 6,149,787 (Andrea W. Chow et al.) issued No. 21, 2000, U.S. Pat. No. 6,149,870 (J. Wallace Parce et al.) issued Nov. 21, 2000, U.S. Pat. No. 6,150,119 (Anne R. Kopf-Sill et al.) issued Nov. 21, 2000, U.S. Pat. No. 6,150,180 (J. Wallace Parce et al.) issued Nov. 21, 2000, U.S. Pat. No. 6,153,073 (Robert S. Dubrow et al.) issued Nov. 28, 2000, U.S. Pat. No. 6,156,181 (J. Wallace Parce et al.) issued Dec. 5, 2000, U.S. Pat. No. 6,167,910 (Calvin Y. H. Chow) issued Jan. 2, 2001, U.S. Pat. No. 6,171,067 (J. Wallace Parce) issued Jan. 9, 2001, U.S. Pat. No. 6,171,850 (Robert Nagle et al.) issued Jan. 9, 2001, U.S. Pat. No. 6,172,353 (Morten J. Jensen) issued Jan. 9, 2001, U.S. Pat. No. 6,174,675 (Calvin Y. H. Chow et al.) issued Jan. 16, 2001, U.S. Pat. No. 6,182,733 (Richard J. McReynolds) issued Feb. 6, 2001, and U.S. Pat. No. 6,186,660 (Anne R. Kopf-Sill et al.) issued Feb. 13, 2001; and published PCT applications, such as, WO 98/00231, WO 98/00705, WO 98/00707, WO 98/02728, WO 98/05424, WO 98/22811, WO 98/45481, WO 98/45929, WO 98/46438, and WO 98/49548, WO 98/55852, WO 98/56505, WO 98/56956, WO 99/00649, WO 99/10735, WO 99/12016, WO 99/16162, WO 99/19056, WO 99/19516, WO 99/29497, WO 99/31495, WO 99/34205, WO 99/43432, WO 99/44217, WO 99/56954, WO 99/64836, WO 99/64840, WO 99/64848, WO 99/67639, WO 00/07026, WO 00/09753, WO 00/10015, WO 00/21666, WO 00/22424, WO 00/26657, WO 00/42212, WO 00/43766, WO 00145172, WO 00/46594, WO 00/50172, WO 00/50642, WO 00/58719, WO 00/060108, WO 00/070080, WO 00/070353, WO 00/072016, WO 00/73799, WO 00/078454, WO 00/102850, and WO 00/114865.

In addition, various other elements are optionally included in the device, such as particle sets, separation gels, antibodies, enzymes, substrates, and the like. These optional elements are used in performing various assays, such as enzyme inhibition assays. For example, in a kinase reaction a product and substrate are typically separated electrophoretically on a separation gel. Cell based microscale assays, e.g., cell based reactions requiring long-incubation times, are also optionally performed in the devices of the invention. Cell-based microscale systems are set forth in Parce et al. “High Throughput Screening Assay Systems in Microscale Fluidic Devices” WO 98/00231 and, e.g., in No. 60/128,643 filed Apr. 4, 1999, entitled “Manipulation of Microparticles In Microfluidic Systems,” by Mehta et al.

Complete integrated systems with fluid handling, signal detection, sample storage and sample accessing are also available. For example WO 98/00231 (supra) provides pioneering technology for the integration of microfluidics and sample selection and manipulation.

Also included in the integrated systems of the invention are sources of sample materials, enzymes, and substrates. These fluidic materials are introduced into the devices by the methods described below.

### Sources of Assay Components and Integration With Microfluidic Formats

Reservoirs or wells are provided in the present invention as sources of buffers, diluents, substrates, enzymes, reagents, samples, and the like. For example, FIG. 3 illustrates various reservoirs, such as substrate well 308, enzyme well 316, waste well 302, and the like. These reservoirs are fluidly coupled to, e.g., channel 350, channel 320, and capillary attachment point 318. For example, samples and/or buffers are optionally added from a microwell plate into the device via a capillary or pipettor channel attached to the device at capillary attachment point 318. The samples are then optionally reacted with other reagents, e.g., substrates and/or enzymes.

Sources of samples, buffers, and reagents, e.g., substrates, enzymes, and the like, are fluidly coupled to the microchannels noted herein in any of a variety of ways. In particular, those systems comprising sources of materials set forth in Knapp et al. “Closed Loop Biochemical Analyzers” (WO 98/45481; PCT/US98/06723) and Parce et al. “High Throughput Screening Assay Systems in Microscale Fluidic Devices” WO 98/00231 and, e.g., in No. 60/128,643 filed Apr. 4, 1999, entitled “Manipulation of Microparticles In Microfluidic Systems,” by Mehta et al. are applicable.

In these systems, a “pipettor channel” (a channel in which components can be moved from a source to a microscale element such as a second channel or reservoir) is temporarily or permanently coupled to a source of material. The source can be internal or external to a microfluidic device comprising the pipettor channel. Example sources include microwell plates, membranes or other solid substrates comprising lyophilized components, wells or reservoirs in the body of the microscale device itself and others.

For example, the source of a cell type, sample, or buffer can be a microwell plate external to the body structure, having at least one well with a sample of interest, e.g., the sample plug and/or buffer plugs of the invention. Alternatively, the source is a well disposed on the surface of the body structure comprising a selected cell type, component, or reagent, a reservoir disposed within the body structure comprising the selected cell type, component, mixture of components, or reagent; a container external to the body structure comprising at least one compartment comprising the selected particle type, component, or reagent, or a solid phase structure comprising the selected cell type or reagent in lyophilized or otherwise dried form.

A loading channel region is optionally fluidly coupled to a pipettor channel with a port external to the body structure. The loading channel can be coupled to an electropipettor channel with a port external to the body structure, a pressure-based pipettor channel with a port external to the body structure, a pipettor channel with a port internal to the body structure, an internal channel within the body structure fluidly coupled to a well on the surface of the body structure, an internal channel within the body structure fluidly coupled to a well within the body structure, or the like.

An integrated microfluidic system of the invention optionally includes a very wide variety of storage elements for storing reagents to be assessed. These include well plates, matrices, membranes and the like. The reagents are stored in liquids (e.g., in a well on a microtiter plate), or in lyophilized form (e.g., dried on a membrane or in a porous matrix), and can be transported to an array component, region, or channel of the microfluidic device using conventional robotics, or using an electropipettor or pressure pipettor channel fluidly coupled to a region or channel of the microfluidic system.

The above devices, systems, features, and components are designed and used according to the methods described below to provide high-throughput screening, e.g., in systems with long incubation or reaction times.

II. Method of Designing a Microfluidic Device Useful for High Throughput Screening in Systems With Long Incubation Times.

To design a microfluidic device for high-throughput screening in systems with long incubation times, the channels are typically configured to provide space for multiple samples to incubate or react such that a first sample is loaded and then incubates while the other samples are being loaded. For example, in a system of parallel channels, one sample or set of samples is optionally loaded into one channel and parked and/or incubated while other parallel channels are loaded. Therefore, one channel loads while the previously loaded channel or channels incubate. Each sample is allowed to incubate until all other samples have been loaded or unloaded. During that time, the desired incubation or reaction time is achieved. If enough other samples are loaded, then by the time loading is completed, the first sample has been incubating, e.g., for about 5 minutes to about 30 minutes. The samples are then serially detected and off-loaded from the channels, providing space in the channels to begin loading again. As each sample is moved into the detection region from, e.g., an incubation channel, another sample is loaded such that it begins incubating or reacting. By iteratively loading, incubating, detecting, and unloading high throughput performance is obtained. For example, rates of about 1 sample per about every 60 seconds or less are obtained.

To provide this type of output, channels are optionally configured to store samples for an appropriate amount of time. The number and length of channels provided and the length of the sample plugs loaded into the channels are varied to achieve selected incubation times. In addition, the diffusion and/or dispersion are typically calculated and taken into account when deciding how many samples are allowed to incubate in a given channel or how long a sample plug is required to be to insure that the samples do not diffuse into each other. Each sample diffuses as it is parked in an incubation channel. The amount of this diffusion/dispersion is typically calculated and an appropriate channel length and sample plug length chosen, e.g., to avoid sample mixing and yet still load enough samples into each channel such that the first sample loaded incubates for the desired incubation time by the time the last sample is loaded. Spacers and/or buffers are optionally used to keep samples separated and/or prevent diffusion of the samples. In addition, flow rates are altered to alter the amount of Taylor dispersion a sample undergoes.

The present invention provides methods of designing devices to provide specified incubation times for a selected number of samples, e.g., in a high-throughput format. The method comprises selecting a desired number of samples. The “desired number of samples” refers to the number of samples stored or incubated in the channels of the device at one time or in one round of samples. For example 40 samples are optionally incubated in four channels that each hold 10 samples. Typically, the number of samples concurrently incubated in a device of the present invention ranges from about 10 to about 1000 samples. More typically, the number of samples ranges from about 20 samples to about 500 samples or from about 40 samples to about 250 samples. Alternatively, the number of samples is about 80 samples to about 150 samples.

The method also comprises selecting a desired incubation time. The “desired incubation time” is the time that a reaction or assay of interest is typically allowed to react, mix, or incubate. For example, a substrate and enzyme in a fluorogenic enzyme inhibition assay typically take about 10 minutes to about 30 minutes to produce a product. Therefore, the components of the reaction are mixed and “parked” or positioned within the channels of the device for an incubation time ranging from about 5 minutes to about 30 minutes.

Another step in the present method involves selecting a sample plug length. The “sample plug length” refers to the length of the sample or the length of channel used by the sample. A sample is loaded into the channel and takes a certain amount of space within that channel. The length of that space is referred to as the sample plug length. A “selected sample plug length” is a length that has been selected based on or taking into account, e.g., the thermal diffusivity of the sample, the Taylor dispersion of the sample, and the desired incubation time. Typically, the sample plug length is selected to be substantially equal to incubation time desired multiplied by the thermal diffusivity/dispersion of the sample for that incubation time or longer. Typical sample plug lengths range from about 500 μm to about 5 mm, more preferably from about 600 μm to about 3 mm, or from about 850 μm to about 1 mm.

Buffer plugs are optionally used to separate samples in the devices of the invention. For example, a buffer plug is loaded into the channel after each sample to avoid mixing of various samples. Buffer plug lengths are selected on the same basis as sample plug lengths, e.g., taking into account the thermal diffusivity/Taylor dispersion of the buffer material, the desired incubation time, and/or the allowable level of mixing between adjacent samples. The buffer plugs typically have the about the same range of lengths or greater since the last buffer spacer loaded is optionally longer than the others to allow for flow pinching.

A plurality of channels is provided in the device based on the above information, e.g., the desired incubation times and selected sample plug length and buffer plug length. The channels are typically interconnected channels that are fluidly coupled to, e.g., each other, waste wells, detection channel regions, sample sources, and the like. The number and length of the channels is chosen taking into account the above information. For example, the “combined channel length” or the sum of the length of all of the channels, e.g., the incubation channels, substantially equals the desired number of samples multiplied by the sample plug length. The combined channel length based on the number of samples and their lengths in the channel provides a channel or channels that are long enough to accommodate all of the samples for the incubation time desired. The combined channel length is typically about 20 mm to about 2000 mm. Alternatively, the combined length is shorter if fewer samples or shorter incubation times are desired, e.g., about 40 mm to about 500 mm or about 50 mm to about 200 mm.

For example, the number of channels selected for the present devices is typically about 2 to about 50 channels. More typically, the number of channels ranges from about 2 to about 30 channels. Preferably, the number of channels ranges from about 4 to about 20 channels or about 6 to about 10 channels. For example a device is optionally designed with two 160 mm parallel incubation channels, for a combined channel length of 320 mm. Alternatively, a device is designed and used in the following methods with four 80 mm channels, providing a combined channel length of 320 mm. In another embodiment, eight 60 mm incubation channels are provided for a combined channel length of 480 mm. For example, a channel system with a combined length of 480 mm optionally holds 80 samples and 80 buffers when each sample plug and buffer plug comprises a length of 3 mm.

A high-throughput sampling rate is optionally selected for the present screening methods and devices. The sampling rate typically ranges from about 60 seconds per sample to about 6 seconds per sample. For example, the sampling rate per sample is optionally 60 seconds or less, 40 seconds or less, 20 seconds or less, 10 seconds or less, 6 seconds or less, or the like. The sampling rate is the rate at which samples are processed thought the device or system. For example, a sampling rate of 60 seconds/sample indicates that one sample is loaded every 60 seconds and/or one sample is detected every 60 seconds. Depending on the channel lengths and number of samples involved, the samples remain in the device anywhere from 5 minutes to an hour between loading and detecting. The sampling rate is optionally selected and adjusted by adjusting, e.g., sample plug length, buffer plug length, loading time per sample, loading time per buffer, loading time per channel, or the like.

To select sample plug lengths and buffer plug lengths, the method optionally comprises calculating thermal diffusivity. “Thermal diffusivity,” as used herein, refers to the amount or distance a sample diffuses in a channel during the incubation and/or reaction time. For example, for a 10 minute incubation, a sample will remain in the channel and diffuse or spread out in the channel for 10 minutes. The amount of diffusion is typically taken into account when selecting a sample plug length or the length of channel space allotted to each sample. For longer incubation times, more diffusion occurs and a longer sample plug is typically allotted for each sample. Therefore more channels or longer channels are selected in the device design.

The amount of diffusion is optionally calculated using the diffusion coefficient (D), which for a small molecule is approximately 3×10⁻⁶ cm²/sec. The amount of diffusion in a microfluidic channel is calculated as \sqrt{2Dt}. Therefore, the amount of diffusion for a 10 minute incubation time is 650 μm and for a 20 minute incubation time, the thermal diffusivity is approximately 850 μm. For more on diffusion see, e.g., Crank, The Mathematics of Diffusion, 2^{nd} Ed. (Oxford Univ. Press 1994). For these levels of diffusion, a typical sample plug length is approximately 3 mm long on the channel and a typical buffer plug length is selected to be about 5 mm long. Therefore, to assay 40 samples in 4 channels (10 samples per channel), each channel is optionally selected to be at least about 80 mm long.

Rates of dispersion of materials within microfluidic systems also affect the sample plug length, e.g., in the same manner as diffusion, described above. As used herein, the term “dispersion” refers to the convection-induced, longitudinal dispersion of material within a fluid medium due to velocity variations across streamlines, e.g., in pressure driven flow systems, electrokinetically driven flow systems around curves and comers, and electrokinetically driven flow systems having non-uniform buffer ionic concentrations, e.g., plugs of high and low salt solutions within the same channel system. For the purposes of the channel systems of the present invention, dispersion is generally defined as that due to the coupling between flow and molecular diffusion, i.e., Taylor dispersion. In this regime, the time-scale for dispersion due to convective transport is long or comparable to the time scale for molecular diffusion in the direction orthogonal to the flow direction. For discussions on dispersion and Taylor dispersion in particular, see, e.g., Taylor et al., Proc. Roy. Soc. London, (1953) 219A:186-203; Aris, Proc. Roy. Soc. London (1956) A235:67-77; Chatwin et al., J. Fluid Mech. (1982) 120:347-358; Doshi et al., Chem. Eng. Sci. (1978) 33:795-804; and Gnell et al., Chem. Eng. Comm. (1987) 58:231-244, each of which is incorporated herein by reference. Channel design optimization in light of dispersion and diffusion of serially introduced reagents is described in “Methods and Software for Designing Microfluidic Devices,” U.S. Ser. No. 09/277,367 filed Mar. 26, 1999 by Chow et al. and in “Optimized High-Throughput Analytical System,” U.S. Ser. No. 09/233,700 filed Jan. 19, 1999 by Kopf-Sill et al., which are incorporated herein by reference.

When multiple samples are introduced into a microfluidic channel, e.g., for a series of assays, more than one sample is optionally flowed through a channel at the same time, e.g., when long incubation times are desired, many different samples are parked or positioned within the channel during the incubation time. These samples diffuse as described above and potentially mix together at some time, e.g., when they are loaded one after the other with no time or space between the sample injections. However, if the samples are spatially separated in the channel, diffusion occurs without mixing the various samples. The calculations described above are optionally used to determine the extent of diffusion and/or dispersion, from which an ideal spacer length is optionally determined.

The present invention provides methods of separating samples with spacers, e.g., buffer plugs, therefore providing rapid and efficient introduction of multiple samples. Spatial separation is obtained in microfluidic channels in a variety of ways, e.g., by separating the samples with high salt fluids and guard bands. See, e.g., U.S. Pat. No. 5,942,443, “High Throughput Screening Assay Systems in Microscale Fluidic Devices,” by Parce et al. The patent describes the use of low ionic strength spacer fluids on either side of a sample plug to aid in electrokinetic pumping of samples through microfluidic channels. These low ionic strength fluids are combined with guard bands or plugs on either end of the sample plug to prevent migration, e.g., electrophoretic migration, of sample elements into the spacer fluid band. Spacers and guard bands are described in more detail, e.g., in U.S. Pat. No. 5,779,868, “Electropipettor and Compensation Means for Electrophoretic Bias,” by Parce et al. See also, “External Material Accession Systems and Methods,” by Chow et al., filed Oct. 12, 1999, which describes alternative spacer materials, e.g., immiscible fluids. In addition, long incubation times are optionally obtained using immiscible fluid spacers between compounds to prevent compound dispersion.

Alternative methods of configuring channels to provide long incubation times include, but are not limited to, configuring the channels to provide a slower flow rate so that samples move through a device at a rate that provides the desired incubation time by the time the sample reaches a detection region. For example, channel resistances may be altered to provide slower flow rates and by-pass channels may be introduced into the device to slow flow rates. Methods of altering channel resistances and bypass channels are described in the various patents and published applications cited herein.

Examples of devices, which are designed according to the above parameters, are described below. These devices are optionally used to perform assays as described below.

III. Examples of Devices for Performing High Throughput Assays With Long Incubation Times.

One embodiment of a device of the present invention is illustrated in FIG. 3. The device makes use of multichannel, e.g., four channels, and multiport control to load samples serially along one channel at a time while samples in other channels undergo long incubations or reactions. As shown, the device comprises a capillary or pipettor channel attached at capillary attachment point 318, which capillary is used to introduce one or more samples or fluidic materials into the device, e.g., from a microwell plate. The one or more samples or fluidic materials optionally comprises different samples or different aliquots of the same sample. Samples are optionally introduced into the system from capillary attachment point 318 and flowed through, e.g., channel region 350 and channels 320, 322, 324, and 326. Additional materials are optionally added to a fluidic material or sample, e.g., from reservoirs 308 and 316, as it flows through main channel region 350. For example, a substrate and enzyme are optionally added to each member of a plurality of samples, e.g., potential enzyme activators, as they flow through channel region 350.

The samples are thus loaded onto the device and parked or positioned within parallel channels 320, 322, 324, and 326, in which they undergo reaction, incubations, or the like for, e.g., about 5 minutes to about 50 minutes. The samples and/or products resulting from the incubation or reaction are then serially unloaded or off-loaded and more samples loaded to continue the long incubation time high-throughput system. Sample, as used herein, typically refers to one or more sample plug. A sample plug includes an initial sample aliquot and any products produced by incubation or reaction of the initial sample aliquot. As they are unloaded, e.g., from the parallel incubation channels, the samples are typically detected, e.g., in detection region 328. Detection of samples includes detection of initial sample plug components and reacted or incubated sample plug components. Detectors are optionally placed proximal to detection region 328 for detecting samples, e.g., as they flow through channels 320, 322, 324, and 326 on the way to reservoirs, e.g., waste wells 302, 304, 310 and 312. The samples in each channel are optionally simultaneously or serially detected. For example, multiple detectors are optionally placed such that detection occurs simultaneously in all of the incubation or analysis channels.

The samples are flowed through the device using, e.g., one or more pressure sources, which are optionally located at reservoirs 302, 304, 310, 312, and the like. The flow of samples through the interconnected channels, e.g., channels 350, 320, 322, 324, and 326, is optionally controlled by an electrokinetic controller coupled to the device. In an electrokinetically controlled device, the samples are flowed through various channels, e.g., fluid flow is switched from one channel to another, e.g., from channel 320 to channel 322, by switching electric fields on and off between appropriately placed nodes. For example an electrokinetic gradient is applied at reservoir 304 and at capillary 318 to flow samples through channel 320. The gradient is optionally switched to reservoir 302 to switch flow to channel 322, reservoir 310 for flow in channel 324 and reservoir 312 for flow in channel 326.

Switching flow between channels is similarly achieved in a pressure controlled system. Pressure is optionally switched between on-chip waste wells, e.g., reservoirs 302, 304, 310, and 312, using, e.g., a multi-source pressure vacuum system. For example, multiple vacuum sources are optionally applied at each of the four waste reservoirs, e.g., reservoirs 302, 304, 310, and 312. The vacuum levels at each well are then varied to control flow through each channel, e.g., to switch flow between different channels.

Alternatively, a single pressure source, e.g., a vacuum source, is plumbed to a manifold of electronically controlled valves, e.g., solenoid valves. Samples are optionally injected into a device via a capillary. Flow is directed into one of several parallel channels, e.g., 4-50 parallel incubation channels. Each channel is typically fluidly coupled to a reservoir, e.g., a waste well, at which point a vacuum source is optionally coupled. The flow is optionally switched between the channels by switching a vacuum source to the appropriate well. Each waste well is plumbed or coupled to an individually controlled vacuum source or to a valve manifold connected to a single vacuum source. For example, in FIG. 3, pressure is optionally applied at well 312 to load samples from a microtiter plate into channel 326. At the same time, pressure, e.g., a partial vacuum, is applied at wells 304, 310, and 302 to equal the existing pressure at the capillary, which is typically under a pressure slightly greater than atmospheric pressure. Application of this equal pressure maintains the samples in channels 320, 322, and 324 in place during the incubation by preventing flow in those channels. In addition, reagent wells, e.g., reservoirs 308 and 316, are optionally placed under a partial vacuum as well to stop flow from these wells during incubation. A second vacuum source is optionally used to maintain an equilibrium pressure on channels containing incubating samples and/or reagent wells. In other embodiments, a vacuum is simultaneously applied at well 304 to draw samples into detection region 328 from channel 320. In this case, samples are loaded into channel 326 concurrently with samples being unloaded from channel 320 for detection. Therefore, when the selected number of samples has been loaded into channel 326 the loading may continue in channel 320 and the unloading from channel 322 by applying pressure at the appropriate waste wells.

In addition reagent wells are optionally included in the devices of the present invention. For example, FIG. 3 comprises reagent wells 308 and 316. The wells are optionally used to feed parallel reaction channels. For example, a substrate and enzyme are optionally added to the samples, e.g., before they have been positioned or parked in the channel for incubation.

Alternative design considerations for the channels of the devices include adjustment of channel dimensions so that transit times, e.g., from capillary inlet 318 to a first reagent mixing point, from the first mixing point to a second mixing point, and from the second mixing point to a detection point, e.g., detection window 328, are the same for all channels in order to simplify the timing of the injection and switching currents.

The use of multiple channels allows for reasonable sample throughput while still permitting long incubation or reaction times. For example, the use of four parallel channels, e.g., 80 mm channels, as illustrated in FIG. 3, allows the injection of one sample about every 20 to about every 24 seconds or less while incubating each reaction mixture for about 15 to about 17 minutes on the device. The use of these devices is described in more detail below.

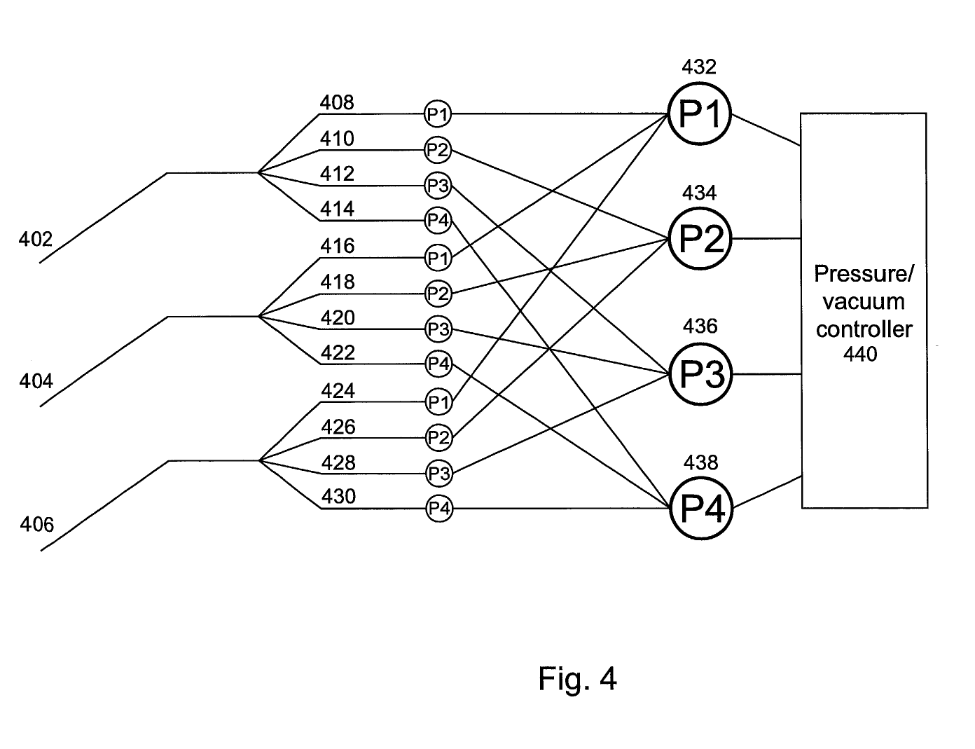

In other embodiments, the use of a multiplexed system of channels connected to one fluid controller is used to increase the amount of samples analyzed in a given period of time, e.g., while decreasing the number of control elements. For example, in FIG. 4, sippers 402, 404 and 406 are each optionally fluidly coupled to one or more sample sources, e.g., microwell plates. The sippers are optionally fluidly coupled to one or more incubation channels, e.g., channels 408, 410, 412, 414, and the like. For example, sipper capillary 404 is fluidly coupled to channels 416, 418, 420, and 422. The incubation and/or analysis channels are optionally coupled to a pressure or fluid control element, e.g., vacuum controller 440. Vacuum controller 440 optionally comprises four discreet fluid control elements, e.g., pressure controllers or vacuums such as 432, 434, 436, and 438. In FIG. 4, one channel from each sipper is connected to pressure control element 432, one channel from each sipper to pressure control element 434, one channel from each sipper to pressure control element 436, and one channel from each sipper to pressure control element 438. Therefore, pressure control element 432 is optionally used to control movement of samples in incubation channels 408, 416, and 424, pressure control element 434 is optionally used to control movement in incubation channels 410, 418, and 426, pressure control element 436 to control movement of fluid in channels 412, 420, and 428 and pressure control element 438 to control movement in incubation channels 414, 422, and 430. Therefore, samples are optionally simultaneously loaded into top channels 408, 416, and 424 and then incubated, e.g., parked or moved slowly through the channels, while samples are simultaneously loaded into middle channels 410, 418, and 426, and so on. For example channels 408, 416, and 424 are optionally simultaneously loaded using pressure control element 432. One pressure control element is therefore optionally used to control sample movement in three different channels. The three sipper configurations are then optionally run simultaneously, thus increasing the number of samples that are analyzed at one time, e.g., using one set of fluid control elements. Alternative design configurations for the multiplexed system of FIG. 4 include channel configurations with more incubation channels, e.g., about 2 to about 50 incubation channels connected to each sipper and channel configurations comprising more sipper systems.

IV. Incubating a Plurality of Samples in a High Throughput System

The above devices, e.g., those designed by the methods provided, are optionally used to provide high throughput screening in systems with long incubation or reaction times, e.g., fluorogenic enzyme assays, binding assays, non-fluorogenic enzyme assays, kinase assays, cell based assays, in-line PCR reactions, and the like. The multiple channels are used to park or store samples while incubating them or reacting them for periods typically ranging from about 5 minutes to about 1 hour. The length of the channels is selected based on the sample plug lengths (which is based on the diffusivity and/or dispersion of the sample and the selected incubation time). The channels are long enough to park at least a portion of the plurality of samples within each channel. One channel is loaded while others are incubated and are detected. The system is run continuously providing high throughput and long incubation times.

For example, about 10 to about 1000 samples are optionally loaded onto a single device or a series of multiplexed sipper devices coupled to a single controller for screening. If the plurality of samples comprises four samples and the device used comprises four channels, then 10 samples are loaded into each of the channels. If the diffusivity of the sample is about 850 μm for 20 minutes, then a typical sample plug is optionally selected to be 3 mm long. A buffer plug to separate the samples is also optionally used, e.g., a 5 mm buffer plug. For a system with four channels and 10 samples of length 3 mm and 10 buffer plugs of about 5 mm each, then each of the four channels is typically about 80 mm.

The plurality of samples is divided into portions and each portion is loaded into a different channel. For example, in a 40 sample, 4 channel system, 10 samples are serially loaded into a first channel, then ten samples are loaded into a second channel, a third channel, a fourth channel (and so on for more channels and more samples in larger systems). When all channels have been loaded, the first samples are then typically unloaded, e.g., into a detection region for detection and/or into a waste reservoir or well.

Each sample is typically loaded into a channel of the device in about 6 seconds to about 60 seconds, preferably in about 20 seconds. Therefore each channel typically takes about 100 to about 600 seconds to load in a system that has 10 samples per channel. Preferably, each channel takes about 100 to about 500 seconds to load or about 200 to about 300 seconds. In a four channel system in which each sample takes about 20 seconds to load, each channel is loaded in about 200 seconds to about 250 seconds, allowing some extra time, e.g., for a longer last buffer plug to accommodate flow pinching. Therefore, the total load time for the system is about 13 to about 17 minutes. The first sample loaded incubates for about 13-17 minutes while the remaining samples are loaded. Therefore, a long incubation time is easily obtained by providing an appropriate channel length and adjusting the load time. The throughput remains high however, because one sample is processed about every 20 seconds to about every 25 seconds. The channel lengths are altered as described above to allow for more samples and the times are adjusted to provide the appropriate incubation times and throughput.

In addition to loading samples, buffers plugs or spacers are also optionally loaded into the channels of the device. For example, a buffer is optionally loaded into a channel after each sample to separate samples form one another and prevent contamination. In addition, the buffer plugs optionally comprise immiscible fluids to decrease diffusion. With decreased diffusion, shorter channels are optionally used or more samples are optionally added per channel. Buffer plug lengths are calculated in the same way as sample plug lengths, e.g., based on thermal diffusivity and/or dispersion of the material. For example a buffer plug is typically 500 μm to 5 mm, preferably 600 μm to 3 mm or 850 μm 1 mm. The last buffer plug loaded or added into a channel or the device is optionally longer, e.g., 500 um to about 10 mm, to allow for flow pinching. When loading buffer plugs, their length is taken into account when determining channel length and load time to produce the desired incubation time.

Using the methods of the present invention, samples are optionally processed in assays with long incubation times in a high throughput manner. The samples are loaded and then incubate while remaining samples are loaded. When all samples have been loaded, e.g., serially, the samples are typically serially flowed through a detection region, starting with the first samples loaded, and detected, e.g., by fluorescence detection. In the detection region the sample plugs, e.g., the sample aliquot and/or one or more reaction product, e.g., fluorescent products, are detected. By the time it is detected, each sample and/or product has typically been incubating from about 5 minutes to about 50 minutes, preferably from about 10 minutes to about 30 minutes. The incubation time depends on the channel length, the number and length of sample plugs and buffer plugs, and the loading time as described above. The loading time and/or detection time provides sampling rates of at least one sample every 60 seconds, about every 40 seconds, about every 20 seconds, about every 10 seconds, or about every 6 seconds or less. As samples are being unloaded to a detection region and then optionally to a waste reservoir, more samples are optionally loaded into the channels to begin the process again. The steps of the method are iteratively repeated to process thousands of samples, e.g., in a day.

V. Instrumentation

Although the devices and systems specifically illustrated herein are generally described in terms of the performance of a few or one particular operation, it will be readily appreciated from this disclosure that the flexibility of these systems permits easy integration of additional operations into these devices. For example, the devices and systems described optionally include structures, reagents and systems for performing virtually any number of operations both upstream and downstream from the operations specifically described herein. Such upstream operations include sample handling and preparation operations, e.g., cell separation, extraction, purification, amplification, cellular activation, labeling reactions, dilution, aliquotting, and the like. Similarly, downstream operations may include similar operations, including, e.g., separation of sample components, labeling of components, assays and detection operations, electrokinetic or pressure-based injection of components into contact with particle sets, or materials released from particle sets, or the like.

In the present invention, materials such as cells, proteins, antibodies, enzymes, substrates, buffers, or the like are optionally monitored and/or detected, e.g., so that the presence of a component of interest can be detected, an activity of a compound can be determined, or an effect of a modulator on, e.g., an enzyme's activity, can be measured. Depending on the label signal measurements, decisions are optionally made regarding subsequent fluidic operations, e.g., whether to assay a particular component in detail to determine, e.g., kinetic information.

The systems described herein generally include microfluidic devices, as described above, in conjunction with additional instrumentation for controlling fluid transport, flow rate and direction within the devices, detection instrumentation for detecting or sensing results of the operations performed by the system, processors, e.g., computers, for instructing the controlling instrumentation in accordance with preprogrammed instructions, receiving data from the detection instrumentation, and for analyzing, storing and interpreting the data, and providing the data and interpretations in a readily accessible reporting format. For example, the systems herein optionally include a valve manifold and a plurality of solenoid valves to control flow switching between channels and/or to control pressure/vacuum levels in the channels, e.g., analysis or incubation channels.

Fluid Direction System

A variety of controlling instrumentation is optionally utilized in conjunction with the microfluidic devices described above, for controlling the transport and direction of fluidic materials and/or materials within the devices of the present invention, e.g., by pressure-based or electrokinetic control.

In the present system, the fluid direction system controls the transport, flow and/or movement of a plurality of samples through the microfluidic device in a high throughput manner. For example, the fluid direction system optionally directs the movement of one or more samples into a first channel, where the samples are optionally incubated. It also optionally directs the simultaneous movement of one or more samples into a detection region. Therefore, the fluid direction system directs the loading and unloading of the plurality of samples in the devices of the invention. The fluid direction system also optionally iteratively repeats these movements to create high throughput screening, e.g., of thousands of compounds.

In addition, the fluid direction system optionally directs the movement of one or more reagent materials, e.g., substrates, enzymes, and the like, from reagent reservoirs, such as reservoirs 308 and 316 in FIG. 3, into a main channel region, such as channel region 350 in FIG. 3, to react with or incubate with, e.g., sample materials. In addition, movement of the sample materials, e.g., incubated or reacted sample materials, through the channels and into a detection region, where they are detected, e.g., by fluorescence, is also controlled by the fluid direction system.

For example, in many cases, fluid transport and direction are controlled in whole or in part, using pressure based flow systems that incorporate external or internal pressure sources to drive fluid flow. Internal sources include microfabricated pumps, e.g., diaphragm pumps, thermal pumps, lamb wave pumps and the like that have been described in the art. See, e.g., U.S. Pat. Nos. 5,271,724, 5,277,556, and 5,375,979 and Published PCT Application Nos. WO 94/05414 and WO 97/02357. As noted above, the systems described herein can also utilize electrokinetic material direction and transport systems. Preferably, external pressure sources are used, and applied to ports at channel termini. More preferably, a single pressure source is used at a main channel terminus. Typically, the pressure source is a vacuum source applied at the downstream terminus of the main channel. These applied pressures, or vacuums, generate pressure differentials across the lengths of channels to drive fluid flow through them. In the interconnected channel networks described herein, differential flow rates on volumes are optionally accomplished by applying different pressures or vacuums at multiple ports, or preferably, by applying a single vacuum at a common waste port and configuring the various channels with appropriate resistance to yield desired flow rates. Example systems are described in U.S. Ser. No. 09/238,467, filed Nov. 28, 1999. In the present invention, for example, vacuum sources optionally apply different pressure levels to various channels to switch flow between the channels. As discussed above, this is optionally done with multiple sources or by connecting a single source to a valve manifold comprising multiple electronically controlled valves, e.g., solenoid valves.

Typically, the controller systems are appropriately configured to receive or interface with a microfluidic device or system element as described herein. For example, the controller and/or detector, optionally includes a stage upon which the device of the invention is mounted to facilitate appropriate interfacing between the controller and/or detector and the device. Typically, the stage includes an appropriate mounting/alignment structural element, such as a nesting well, alignment pins and/or holes, asymmetric edge structures (to facilitate proper device alignment), and the like. Many such configurations are described in the references cited herein.

The controlling instrumentation discussed above is also optionally used to provide for electrokinetic injection or withdrawal of material downstream of the region of interest to control an upstream flow rate. The same instrumentation and techniques described above are also utilized to inject a fluid into a downstream port to function as a flow control element.

Detector

The devices herein optionally include signal detectors, e.g., which detect fluorescence, phosphorescence, radioactivity, pH, charge, absorbance, luminescence, temperature, magnetism, color, or the like. Fluorescent and chemiluminescent detection are especially preferred, for example in fluorogenic enzyme assays.

The detector(s) optionally monitors one or more of a plurality of signals from one or more detection regions of the device, e.g., detection regions 328 in FIG. 3. For example, the detector optionally monitors an optical signal that corresponds to a labeled component, such as a labeled antibody or protein located, e.g., in detection region 328. In one embodiment, the detection region spans multiple main channels and one detector, positioned proximal to the detection region, is used to detect signals from all channels concurrently or serially. For example, in FIG. 3, four separate but interconnected channels are proximal to the detection region 328. Each channel is used to incubate, e.g., a plurality of samples or a portion of the samples. The samples are serially transported through the detection region. For example, ten samples in channel 320 are optionally incubated for 10 minutes and then flowed through the detection region, e.g., for fluorescence detection, and then to a waste well, e.g., waste well 304. The samples from channel 322, which have been incubating while those from channel 320 were detected and unloaded, are then flowed through the detection region and detected, and so on for the rest of the four channels. In addition samples are loaded into each channel as samples from that channel are detected and unloaded.

Examples of detection systems useful in the present invention include optical sensors, temperature sensors, pressure sensors, pH sensors, conductivity sensors, and the like. Each of these types of sensors is readily incorporated into the microfluidic systems described herein. In these systems, such detectors are placed either within or adjacent to the microfluidic device or one or more channels, chambers or conduits of the device, such that the detector is within sensory communication with the device, channel, or chamber. The phrase “proximal,” to a particular element or region, as used herein, generally refers to the placement of the detector in a position such that the detector is capable of detecting the property of the microfluidic device, a portion of the microfluidic device, or the contents of a portion of the microfluidic device, for which that detector was intended. For example, a pH sensor placed in sensory communication with a microscale channel is capable of determining the pH of a fluid disposed in that channel. Similarly, a temperature sensor placed in sensory communication with the body of a microfluidic device is capable of determining the temperature of the device itself.

Particularly preferred detection systems include optical detection systems for detecting an optical property of a material within the channels and/or chambers of the microfluidic devices that are incorporated into the microfluidic systems described herein. Such optical detection systems are typically placed adjacent to a microscale channel of a microfluidic device, and are in sensory communication with the channel via an optical detection window that is disposed across the channel or chamber of the device. Optical detection systems include systems that are capable of measuring the light emitted from material within the channel, the transmissivity or absorbance of the material, as well as the material's spectral characteristics. Example detectors include photo multiplier tubes, a CCD array, a scanning detector, a galvo-scanner or the like. For example, in preferred aspects, a fluorescence, chemiluminescence or other optical detector is used in the assay. Proteins, antibodies, or other components which emit a detectable signal can be flowed past the detector, or, alternatively, the detector can move relative to an array to determine protein position (or, the detector can simultaneously monitor a number of spatial positions corresponding to channel regions, e.g., as in a CCD array).

In preferred aspects, the detector measures an amount of light emitted from the material, such as a fluorescent or chemiluminescent material. As such, the detection system will typically include collection optics for gathering a light based signal transmitted through the detection window, and transmitting that signal to an appropriate light detector. Microscope objectives of varying power, field diameter, and focal length are readily utilized as at least a portion of this optical train. The light detectors are optionally photodiodes, avalanche photodiodes, photomultiplier tubes, diode arrays, or in some cases, imaging systems, such as charged coupled devices (CCDs) and the like. In preferred aspects, photodiodes are utilized, at least in part, as the light detectors. The detection system is typically coupled to a computer (described in greater detail below), via an analog to digital or digital to analog converter, for transmitting detected light data to the computer for analysis, storage and data manipulation.

In the case of fluorescent materials such as labeled cells, the detector typically includes a light source which produces light at an appropriate wavelength for activating the fluorescent material, as well as optics for directing the light source through the detection window to the material contained in the channel or chamber. The light source can be any number of light sources that provides an appropriate wavelength, including lasers, laser diodes and LEDs. Other light sources are required for other detection systems. For example, broad band light sources are typically used in light scattering/transmissivity detection schemes, and the like. Typically, light selection parameters are well known to those of skill in the art.

The detector can exist as a separate unit, but is preferably integrated with the controller system, into a single instrument. Integration of these functions into a single unit facilitates connection of these instruments with a computer (described below), by permitting the use of few or a single communication port(s) for transmitting information between the controller, the detector and the computer. Integration of the detection system with a computer system typically includes software for converting detector signal information into assay result information, e.g., concentration of a substrate, concentration of a product, presence of a compound of interest, or the like.

Computer

As noted above, either or both of the fluid direction system and/or the detection system are coupled to an appropriately programmed processor or computer which functions to instruct the operation of these instruments in accordance with preprogrammed or user input instructions, receive data and information from these instruments, and interpret, manipulate and report this information to the user. As such, the computer is typically appropriately coupled to one or both of these instruments (e.g., including an analog to digital or digital to analog converter as needed).

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing the operation of the fluid direction and transport controller to carry out the desired operation.

For example, the computer is optionally used to direct a fluid direction system to control fluid flow, e.g., through a variety of interconnected channels. The fluid direction system optionally directs the movement of at least a first member of the plurality of samples into a first member of the plurality of channels concurrent with directing the movement of at least a second member of the plurality of samples into the one or more detection channel regions. The fluid direction system also directs the movement of at least a first member of the plurality of samples into the plurality of channels concurrent with incubating at least a second member of the plurality of samples. It also directs movement of at least a first member of the plurality of samples into the one or more detection channel regions concurrent with incubating at least a second member of the plurality of samples.

By coordinating channel switching, the system directs the movement of at least one member of the plurality of samples into the plurality of channels and/or one member into a detection region about every 60 seconds or less, about every 40 seconds or less, about every 30 seconds or less, about every 10 seconds or less, or about every 6 seconds or less. Each sample, with appropriate channel switching as described above, remains in the plurality of channels between about 5 minutes and about 50 minutes. For example the samples optionally remain in the channels for a selected incubation time of, e.g., 20 minutes.

The computer then receives the data from the one or more sensors/detectors included within the system, and interprets the data, either provides it in a user understood format, or uses that data to initiate further controller instructions, in accordance with the programming, e.g., such as in monitoring and control of flow rates, temperatures, applied voltages, and the like.

In the present invention, the computer typically includes software for the monitoring and control of materials in the channels. For example, the software directs channel switching to control and direct flow as described above. Additionally the software is optionally used to control electrokinetic or pressure-modulated injection or withdrawal of material. The injection or withdrawal is used to modulate the flow rate as described above. The computer also typically provides instructions, e.g., to the controller or fluid direction system for switching flow between channels to achieve the high throughput format discussed above.

In addition, the computer optionally includes software for deconvolution of the signal or signals from the detection system. For example, the deconvolution distinguishes between two detectably different spectral characteristics that were both detected, e.g., when a substrate and product comprise detectably different labels.

Example Integrated System

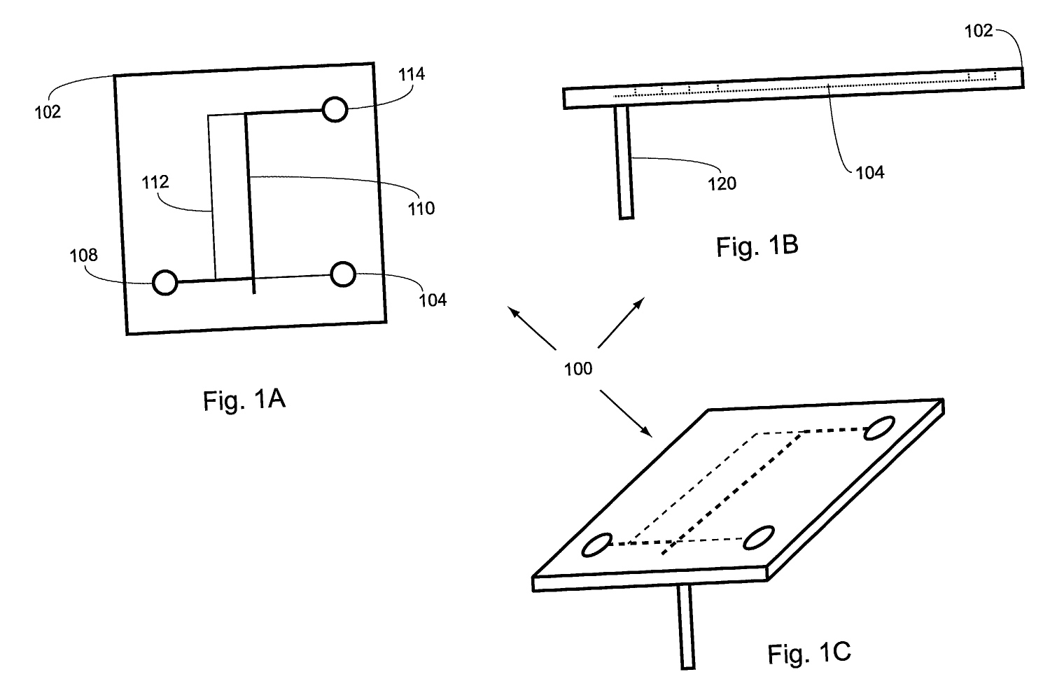

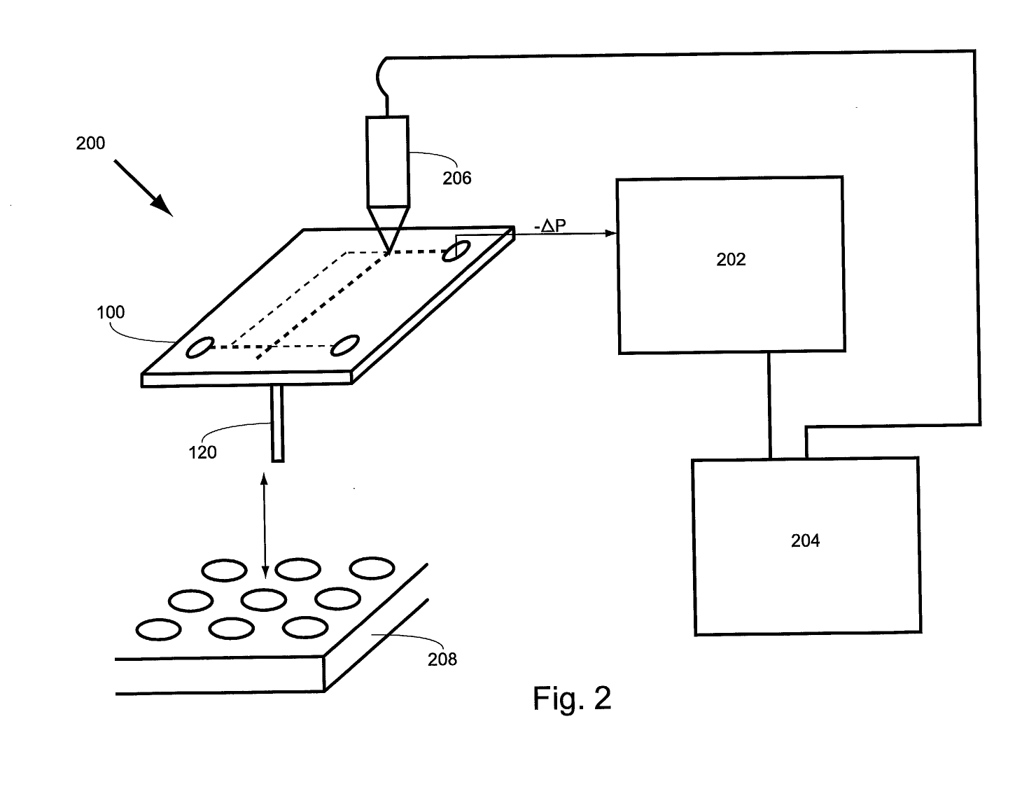

FIG. 1, Panels A, B, and C and FIG. 2 provide additional details regarding example integrated systems that are optionally used to practice the methods herein. As shown, body structure 102 has parallel channels 110 and 112 disposed therein. A sample or a plurality of samples is optionally flowed from pipettor channel 120 towards reservoir 114, e.g., by applying a vacuum at reservoir 114 (or another point in the system) or by applying appropriate voltage gradients. Alternatively, a vacuum is applied at reservoirs 108, 104 or through pipettor channel 120. For example, 20 samples are optionally serially flowed through pipettor channel 120 into channel 112 and positioned there to incubate for twenty minutes. While they remain in channel 112, 20 more samples are serially loaded into channel 110. When loading is completed in channel 110, the first sample loaded into channel 112 has optionally been incubating for twenty minutes or some other selected incubation time, e.g., substantially equal to the time used to load all of the samples. The samples in channel 112 are then optionally unloaded or offloaded, e.g., into reservoir 114, and the process is optionally iteratively repeated to provide high-throughout screening with long incubation times.

Additional materials, such as buffer solutions, substrate solutions, enzyme solutions, and the like, as described above, are optionally flowed from wells 108 or 104 into channel 110 and 112. Flow from these wells is optionally performed by modulating fluid pressure, or by electrokinetic approaches as described (or both). The arrangement of channels depicted in FIG. 1 is only one possible arrangement out of many which are appropriate and available for use in the present invention. Alternatives are provided in FIGS. 3 and 4. Additional alternatives can be devised, e.g., by combining the microfluidic elements described herein, e.g., different channel numbers and lengths, with other microfluidic devices described in the patents and applications referenced herein. Furthermore the elements of FIGS. 3 and 4 are optionally recombined to provide alternative configurations.

Samples and materials are optionally flowed from the enumerated wells or from a source external to the body structure. As depicted, the integrated system optionally includes pipettor channel 120, e.g., protruding from body 102, for accessing a source of materials external to the microfluidic system. Typically, the external source is a microtiter dish or other convenient storage medium. For example, as depicted in FIG. 2, pipettor channel 120 can access microwell plate 208, which includes sample materials, buffers, immiscible fluids, spacers, substrate solutions, enzyme solutions, and the like, in the wells of the plate.

Detector 206 is in sensory communication with channels 112 and 110, detecting signals resulting, e.g., from labeled materials flowing through the detection region. Therefore, before unloading the samples into reservoir 114, the samples are detected as they are removed from the channels, e.g., unloaded from channel 112. Detector 206 is optionally coupled to any of the channels or regions of the device where detection is desired. Detector 206 is operably linked to computer 204, which digitizes, stores, and manipulates signal information detected by detector 206, e.g., using any of the instructions described above, e.g., or any other instruction set, e.g., for determining concentration, molecular weight or identity, or the like.

Fluid direction system 202 controls voltage, pressure, or both, e.g., at the wells of the systems or through the channels of the system, or at vacuum couplings fluidly coupled to channel 112 or other channel described above. Optionally, as depicted, computer 204 controls fluid direction system 202. The computer therefore controls the direction of fluids through the channels and the switching of pressure or gradients across the channels and/or valves, e.g., solenoid valves, to control fluid flow. In one set of embodiments, computer 204 uses signal information to select further parameters for the microfluidic system. For example, upon detecting the presence of a component of interest in a sample from microwell plate 208, the computer optionally directs addition of a potential modulator of component of interest into the system.

Kits

Generally, the microfluidic devices described herein are optionally packaged to include reagents for performing the device's preferred function. For example, the kits can include any of microfluidic devices described along with assay components, reagents, sample materials, proteins, antibodies, enzymes, substrates, control materials, spacers, buffers, immiscible fluids, or the like. Such kits also typically include appropriate instructions for using the devices and reagents, and in cases where reagents are not predisposed in the devices themselves, with appropriate instructions for introducing the reagents into the channels and/or chambers of the device. In this latter case, these kits optionally include special ancillary devices for introducing materials into the microfluidic systems, e.g., appropriately configured syringes/pumps, or the like (in one embodiment, the device itself comprises a pipettor element, such as an electropipettor for introducing material into channels and chambers within the device). In the former case, such kits typically include a microfluidic device with necessary reagents predisposed in the channels/chambers of the device.

Generally, such reagents are provided in a stabilized form, so as to prevent degradation or other loss during prolonged storage, e.g., from leakage. A number of stabilizing processes are widely used for reagents that are to be stored, such as the inclusion of chemical stabilizers (i.e., enzymatic inhibitors, microbicides/bacteriostats, anticoagulants), the physical stabilization of the material, e.g., through immobilization on a solid support, entrapment in a matrix (i.e., a gel), lyophilization, or the like. Kits also optionally include packaging materials or containers for holding microfluidic device, system or reagent elements.

While the foregoing invention has been described in some detail for purposes of clarity and understanding, it will be clear to one skilled in the art from a reading of this disclosure that various changes in form and detail can be made without departing from the true scope of the invention and the invention can be put to a number of different uses. For example, all the techniques and apparatus described above may be used in various combinations. All publications and patent documents cited in this application are incorporated by reference in their entirety for all purposes to the same extent as if each individual publication or patent document were individually so denoted.

What is claimed is:

1. A method of incubating a plurality of samples in a high-throughput microfluidic system, the method comprising:
(i) loading the plurality of samples into a plurality of channels;
(ii) incubating each member of the plurality of samples in the plurality of channels for at least about 5 minutes; and,
(iii) flowing the plurality of samples through one or more detection channel regions;
wherein at least one member of the plurality of samples is loaded into at least one of the plurality of channels about every 60 seconds or less, thereby achieving a long incubation time for substantially all of the samples in the plurality of samples, while concomitantly processing the plurality of samples in a high-throughput format.

2. The method of claim 1, wherein the plurality of samples comprises between about 10 and about 1000 samples.

3. The method of claim 1, wherein the plurality of samples comprises between about 20 and about 500 samples.

4. The method of claim 1, wherein the plurality of samples comprises between about 40 and about 250 samples.

5. The method of claim 1, wherein the plurality of samples comprises between about 80 and about 150 samples.

6. The method of claim 2, wherein the plurality of samples comprises about 40 samples.

7. The method of claim 1, wherein each member of the plurality of samples comprises a selected sample plug length.

8. The method of claim 7, comprising selecting the sample plug length to account for the thermal diffusivity or dispersion of the sample.

9. The method of claim 8, wherein the sample plug length for each member of the plurality of samples comprises between about 500 μm and about 5 mm in length.

10. The method of claim 9, wherein the sample plug length comprises about 600 μm to about 3 mm.

11. The method of claim 9, wherein the sample plug length comprises about 850 μm to about 1 mm.

12. The method of claim 1, comprising loading at least one member of the plurality of samples into the plurality of channels at least about every 60 seconds or less, about every 40 seconds or less, about every 20 seconds or less, about every 10 seconds or less, or about every 6 seconds or less.

13. The method of claim 1, comprising dividing the plurality of samples into a plurality of portions and loading each of the portions into a different member of the plurality of channels.

14. The method of claim 13, comprising dividing the plurality of samples into four portions and loading the portions into four channels.

15. The method of claim 14, comprising loading each of the four portions into one of the four channels in between about 100 seconds and about 500 seconds.

16. The method of claim 15, comprising loading each of the four portions into one of the four channels in about 200 seconds to about 300 seconds.

17. The method of claim 1, comprising: loading a plurality of buffers into the plurality of channels, wherein each member of the plurality of samples comprises a selected sample plug length.

18. The method of claim 17, comprising loading a member of the plurality of buffers into the plurality of channels after loading each member of the plurality of samples into the plurality of channels.

19. The method of claim 17, wherein each member of the plurality of buffers comprises a selected buffer plug length.

20. The method of claim 19, comprising selecting the buffer plug length to account for the thermal diffusivity or dispersion of the buffer.

21. The method of claim 19, wherein the buffer plug length for each member of the plurality of buffers comprises about 500 μm to about 5 mm.

22. The method of claim 21, wherein the buffer plug length comprises about 600 μm to about 3 mm.

23. The method of claim 21, wherein the buffer plug length comprises about 850 μm to about 1 mm.

24. The method of claim 1, wherein the plurality of channels comprises about 2 to about 50 channels.

25. The method of claim 1, wherein the plurality of channels comprises about 2 to about 30 channels

26. The method of claim 1, wherein the plurality of channels comprises about 4 to about 20 channels.

27. The method of claim 1, wherein the plurality of channels comprises about 6 to about 10 channels.

28. The method of claim 7, comprising providing the plurality of channels to comprise a combined channel length, which combined channel length is at least as long as the number of members in the plurality of samples multiplied by the selected sample plug length.

29. The method of claim 19, comprising providing the plurality of channels to comprise a combined channel length, which combined channel length is at least as long as the number of members in the plurality of samples multiplied by the sample plug length and the buffer plug length.

30. The method of claim 1, wherein each member of the plurality of channels comprises a length between about 20 mm and about 2000 mm.

31. The method of claim 30, wherein each member of the plurality of channels comprises a length between about 40 mm and about 500 mm

32. The method of claim 30, wherein each member of the plurality of channels comprises a length between about 40 mm and about 200 mm.

33. The method of claim 30, wherein the plurality of channels comprises two channels, which two channels each comprise a length of about 100 mm to about 200 mm.

34. The method of claim 33, wherein each of the two channels comprises a length of about 160 mm.

35. The method of claim 30, wherein the plurality of channels comprises four channels, which four channels each comprise a length of about 50 mm to 100 mm.

36. The method of claim 35, wherein each of the four channels comprises a length of about 80 mm.

37. The method of claim 30, wherein the plurality of channels comprises eight channels, which eight channels each comprise a length of about 50 mm to about 100 mm.

38. The method of claim 37, wherein each of the eight channels comprises a length of about 80 mm.

39. The method of claim 1, comprising incubating substantially each member of the plurality of samples between about 5 minutes and about 50 minutes.

40. The method of claim 1, comprising incubating substantially each member of the plurality of samples between about 10 minutes and about 30 minutes.

41. The method of claim 1, comprising providing a plurality of fluid control elements fluidly coupled to the plurality of channels.

42. The method of claim 41, further comprising fluidly coupling each member of the plurality of channels to at least one fluid control element.

43. The method of claim 41, wherein the plurality of fluid control elements comprises a plurality of pressure sources.

44. The method of claim 43, wherein the pressure sources comprise vacuum sources.

45. The method of claim 41, wherein the plurality of fluid control elements comprises a plurality of electrokinetic controllers.

46. The method of claim 1, comprising providing a single fluid control element coupled to the plurality of channels.

47. The method of claim 46, wherein the single fluid control element comprises a single pressure source.

48. The method of claim 47, wherein the pressure source comprises a vacuum source.

49. The method of claim 1, comprising iteratively repeating steps (i) through (iii).

50. The method of claim 1, comprising loading at least a first member of the plurality of samples into the plurality of channels concurrent with flowing at least a second member of the plurality of samples through the one or more detection channel regions.

51. The method of claim 1, comprising incubating at least a first member of the plurality of samples concurrent with loading at least a second member of the plurality of samples and flowing at least a third member of the plurality of samples through the one or more detection channel regions.

52. The method of claim 1, comprising detecting the plurality of samples in the one or more detection channel regions.

53. The method of claim 52, wherein detecting comprising fluorescently detecting the plurality of samples.

54. The method of claim 52, comprising detecting at least one member of the plurality of samples about every 60 seconds or less, about every 40 seconds or less, about every 20 seconds or less, about every 10 seconds or less, or about every 6 seconds or less.

55. A microfluidic device, the device comprising:
(i) at least one sample source;
(ii) a plurality of channels fluidly coupled to the at least one sample source; and,
(iii) one or more detection channel regions, fluidly coupled to the plurality of channels;
wherein the plurality of channels has a combined channel length substantially equal to a selected number of samples multiplied by a selected sample plug length and is structurally configured to provide an incubation time of at least about 5 minutes with a throughput of 1 sample about every 60 seconds or less.

56. The device of claim 55, further comprising a fluid control element, which fluid control element loads at least one sample about every 60 seconds or less, about every 40 seconds or less, about every 20 seconds or less, about every 10 seconds or less, or about every 6 seconds or less.

57. A high throughput microfluidic system for achieving long incubations, the system comprising:
(i) a microfluidic device comprising:
(a) at least one sample source;
(b) a plurality of channels fluidly coupled to the at least one sample source; and,
(c) one or more detection channel regions fluidly coupled to the plurality of channels;
wherein the plurality of channels comprise a combined length, which combined length substantially equals a selected number of samples multiplied by a selected sample plug length;
(ii) a fluid direction system, wherein during operation of the system the fluid direction system directs movement of a plurality of samples into the plurality of channels and movement of the plurality of samples into the one or more detection channel regions, wherein the fluid direction system directs the movement of at least one member of the plurality of samples into the plurality of channels about every 60 seconds or less; and,
(iii) a detection system positioned proximal to the one or more detection channel regions.

58. The system of claim 57, wherein the sample source comprises a plurality of samples.

59. The system of claim 57, wherein the sample plug length takes into account the thermal diffusivity of the sample.

60. The system of claim 57, wherein the sample plug length for each member of the plurality of samples comprises between about 500 μm and about 5 mm in length.

61. The system of claim 60, wherein the sample plug length comprises between about 600 μm and about 3 mm in length.

62. The system of claim 60, wherein the sample plug length comprises between about 850 μm and about 1 mm in length.

63. The system of claim 57, wherein the device further comprises one or more buffer sources.

64. The system of claim 63, wherein the plurality of buffer sources comprises a plurality of buffers, which buffers comprise a selected buffer plug length.

65. The system of claim 64, wherein the buffer plug length takes into account the thermal diffusivity or dispersion of the buffer.

66. The system of claim 64, wherein the buffer plug length for each member of the plurality of samples comprises between about 500 μm and about 5 mm in length.

67. The system of claim 66, wherein the buffer plug length comprises about 600 μm to about 3 mm.

68. The system of claim 66, wherein the buffer plug length comprises about 850 μm to about 1 mm.

69. The system of claim 57, wherein the plurality of channels comprises between about 2 and about 50 channels.

70. The system of claim 69, wherein the plurality of channels comprises about 2 to about 30 channels.

71. The system of claim 69, wherein the plurality of channels comprises about 4 to about 20 channels.

72. The system of claim 69, wherein the plurality of channels comprises about 6 to about 10 channels.

73. The system of claim 57, wherein each member of the plurality of channels comprises a length between about 20 mm and about 2000 mm.

74. The system of claim 73, wherein each member of the plurality of channels comprises a length between about 40 mm and about 500 mm.

75. The system of claim 73, wherein each member of the plurality of channels comprises a length between about 40 mm and about 200 mm.

76. The system of claim 64, wherein the combined length of the plurality of channels is substantially equal to the selected number of samples multiplied by the sample plug length and the buffer plug length.

77. The system of claim 73, wherein the plurality of channels comprises four channels, which four channels each comprise a length of about 50 mm to about 200 mm.

78. The system of claim 73, wherein the plurality of channels comprises four channels, which four channels each comprise a length of about 80 mm to about 100 mm.

79. The system of claim 73, wherein the plurality of channels comprises eight channels, which eight channels each comprise a length of about 50 mm to about 200 mm.

80. The system of claim 73, wherein the plurality of channels comprises eight channels, which eight channels each comprise a length of about 80 mm to about 100 mm.

81. The system of claim 73, wherein the plurality of channels comprises two channels, which two channels each comprise a length of about 100 mm to about 200 mm.

82. The system of claim 73, wherein the plurality of channels comprises two channels, which two channels each comprise a length of about 160 mm.

83. The system of claim 57, wherein the plurality of samples comprises between about 10 and about 1000 samples.

84. The system of claim 83, wherein the plurality of samples comprises between about 20 to about 500 samples.

85. The system of claim 83, wherein the plurality of samples comprises between about 40 to about 250 samples.

86. The system of claim 83, wherein the plurality of samples comprises between about 80 to about 150 samples.

87. The system of claim 83, wherein the plurality of samples comprises about 40 samples.

88. The system of claim 85, wherein the plurality of channels comprises about 4 channels and the plurality of samples comprises about 40 samples, wherein about 10 samples are flowed into each of the four channels.

89. The system of claim 57, wherein the fluid direction system comprises one or more fluid control elements.

90. The system of claim 89, wherein the one or more fluid control elements comprise one or more pressure sources.

91. The system of claim 90, wherein the one or more pressure sources comprise vacuum sources.

92. The system of claim 89, wherein the one or more fluid control elements comprise one or more electrokinetic controllers.

93. The system of claim 89, wherein each member of the plurality of channels is fluidly coupled to at least one of the one or more fluid control elements.

94. The system of claim 57, wherein the fluid direction system comprises a single fluid control element, which control element is coupled to the plurality of channels.

95. The system of claim 94, wherein the single fluid control element comprises a single pressure source.

96. The system of claim 95, wherein the single pressure source comprises a vacuum source.

97. The system of claim 57, wherein during operation of the system, the fluid direction system directs the movement of at least a first member of the plurality of samples into a first member of the plurality of channels concurrent with directing the movement of at least a second member of the plurality of samples into the one or more detection channel regions.

98. The system of claim 57, wherein during operation of the system, the fluid direction system directs the movement of at least a first member of the plurality of samples into the plurality of channels concurrent with incubating at least a second member of the plurality of samples.

99. The system of claim 57, wherein during operation of the system, the fluid direction system directs the movement of at least a first member of the plurality of samples into the one or more detection channel regions concurrent with incubating at least a second member of the plurality of samples.

100. The system of claim 57, wherein during operation of the system, the fluid direction system directs movement of at least one member of the plurality of samples into the plurality of channels about every 60 seconds or less, about every 40 seconds or less, about every 30 seconds or less, about every 10 seconds or less, or about every 6 seconds or less.

101. The system of claim 57, wherein during operation of the system, the fluid direction system directs movement of a buffer into the plurality of channels after movement of a member of the plurality of samples into the plurality of channels.

102. The system of claim 101, wherein during operation of the system, the fluid direction system directs movement of a buffer into the plurality of channels after movement of each member of the plurality of samples into the plurality of channels.

103. The system of claim 57, wherein during operation of the system, the fluid direction system directs movement of a member of the plurality of samples into the one or more detection channel regions about every 60 seconds or less, about every 40 seconds or less, about every 20 seconds or less, about every 10 seconds or less, or about every 6 seconds or less.

104. The system of claim 57, wherein during operation of the system, substantially each member of the plurality of samples remains in the plurality of channels between about 5 minutes and about 50 minutes.

105. The system of claim 57, wherein during operation of the system, substantially each member of the plurality of samples remains in the plurality of channels between about 10 minutes and about 30 minutes.

106. The system of claim 57, wherein during operation of the system, substantially each member of the plurality of samples remains in the plurality of channels for about 15 minutes and wherein the fluid direction system directs movement of at least one member of the plurality of samples into the one or more detection channel regions about every 30 seconds or less.

107. The system of claim 57, wherein the detection system comprises one or more detectors positioned proximal to at least one of the one or more detection channel regions.

108. The system of claim 57, wherein the detection system comprises one or more detectors positioned proximal to substantially all of the one or more detection channel regions.

109. The system of claim 57, wherein the one or more detectors comprise fluorescent detectors.

110. A method of designing a microfluidic device, the method comprising:
(i) selecting a desired number of samples for screening by the microfluidic device;
(ii) selecting a desired incubation time for one or more reactions for the desired number of samples;
(iii) selecting a sample plug length for each of the samples in a channel, which sample plug length is at least as long as the incubation time multiplied by the thermal diffusivity of the samples; and,
(iv) providing a plurality of interconnected channels, which channels are fluidly coupled to at least one sample source and at least one detection region, which plurality of interconnected channels have a combined length substantially equal to the desired number of samples multiplied by the sample plug length.

111. The method of claim 110, comprising selecting a desired number of samples between about 10 samples and about 1000 samples.

112. The method of claim 110, comprising selecting a desired number of samples between about 20 samples and about 500 samples.

113. The method of claim 110, comprising selecting a desired number of samples between about 40 samples and about 250 samples.

114. The method of claim 110, comprising selecting a desired number of samples between about 80 samples and about 150 samples.

115. The method of claim 110, comprising selecting a desired incubation time between about 5 minutes and about 50 minutes.

116. The method of claim 110, comprising selecting a desired incubation time between about 10 minutes and about 30 minutes.

117. The method of claim 110, comprising selecting a sample plug length between about 500 μm and about 5 mm.

118. The method of claim 110, comprising selecting a sample plug length between about 600 μm and about 3 mm.

119. The method of claim 110, comprising selecting a sample plug length between about 850 μm and about 1 mm.

120. The method of claim 110, wherein the plurality of interconnected channels comprises between about 2 and about 50 channels.

121. The method of claim 110, wherein the plurality of interconnected channels comprises between about 2 and about 30 channels.

122. The method of claim 110, wherein the plurality of interconnected channels comprises between about 4 and about 20 channels.

123. The method of claim 110, wherein the plurality of interconnected channels comprises between about 6 and about 10 channels.

124. The method of claim 110, wherein each member of the plurality of interconnected channels comprises a length between about 20 mm and about 2000 mm.

125. The method of claim 110, wherein each member of the plurality of interconnected channels comprises a length between about 40 mm and about 500 mm.

126. The method of claim 110, wherein each member of the plurality of interconnected channels comprises a length between about 40 mm and about 200 mm.

127. The method of claim 110, further comprising selecting a sampling rate, which sampling rate is between about 60 seconds per sample and about 6 seconds per sample.

128. The method of claim 110, further comprising selecting a buffer plug length, which buffer plug length is between about 600 μm and about 5 mm, wherein the plurality of interconnected channels have a combined length substantially equal to the desired number of samples multiplied by the sample plug length and the buffer plug length.

129. The method of claim 110, further comprising calculating the thermal diffusivity of the sample to determine the sample plug length.
